(19) United States
(12) Patent Application Publication
(10) Pub. No.: US 2010/0143258 A1
(43) Pub. Date: Jun. 10, 2010

(54) TUMOR MARGIN IMAGING AGENTS

(75) Inventors: Anup Sood, Clifton Park, NY (US); Jason William Castle, Esperance, NY (US); Evelina Roxana Loghin, Rexford, NY (US); Natalie Anne Staples, Albany, NY (US); Kenneth Michael Fish, Clifton Park, NY (US); Randall Lee Carter, Clifton Park, NY (US); Brian Duh-Lan Lee, Rexford, NY (US); Kathleen Bove, Ballston Lake, NY (US)

Correspondence Address: GENERAL ELECTRIC COMPANY;GLOBAL RESEARCH, ONE RESEARCH CIRCLE, PATENT DOCKET RM. BLDG. K1-4A59, NISKAYUNA, NY, 12309, US

(73) Assignee: GENERAL ELECTRIC COMPANY, SCHENECTADY, NY (US)

(21) Appl. No.: 12/328,802

(22) Filed: Dec. 5, 2008

(51) Int. Cl. A61K 49/00; C07K 2/00

(52) U.S. Cl. 424/9.6; 530/345

(57) ABSTRACT

In one aspect, the present invention provides a functionalized monodisperse polylysine comprising a linear monodisperse polylysine chain comprising constituent lysine monomer residues containing appended C₄-C₂₄ polyethylene glycol groups and at least one appended fluorescent dye moiety is provided.

### CROSS REFERENCE TO RELATED APPLICATIONS

This application is related to a U.S. patent application Ser. No. 12/259,944, entitled “TUMOR MARGIN AND DYE INTAKE MEASUREMENT TOOL”, filed Oct. 28, 2008 which is herein incorporated by reference.

### BACKGROUND

The invention relates to functionalized polylysine compositions derived from a linear monodisperse polylysine compound. Further, the present disclosure relates to a method of making the functionalized polylysine composition and relates to a method of using the polylysine composition and related articles comprising the polylysine composition.

In the United States, one in three persons will develop cancer in their lifetime. There are three major treatments for cancer including chemotherapy, radiation and surgery. Curative surgery is the primary treatment for most cancers and can be highly effective with or without additional treatment modalities especially for early stages of cancer. Approximately 60-70% of cancer patients undergo surgical removal of tumor. Surgery is also used for diagnosis, staging and management of complications caused by tumor growth. Despite major advancements in diagnosis and treatment, prognosis is generally associated with success of the original surgery. Additionally, although the surgery is the first line treatment, there are no standardized practices.

With surgery, patient outcome is dependent upon tumor size, pathology of the tumor, and identifiable tumor margins. Tumor margins are critical in the fact that any residual tumor cells that are left can lead to local recurrence. Yet the definition of clear or negative tumor margin differs not only across continents but also within US.

Another factor that impacts the likelihood of local recurrence is the skill of the surgeon performing the tumor resection. One reason surgical treatment fails in the early stages of cancer is because the whole tumor was not removed (lack of clear margins). The current practice of tumor resection at most treatment facilities is guided by visual inspection and palpitation. However cancer tissue is often difficult to distinguish from normal tissue or is too small to feel by hand. Experience, therefore, plays a major role in the success of surgery. In fact, studies demonstrating the effect of a surgeon's experience on the outcome of different surgical procedures (including tumor resection) have been published.

It is believed that, imaging methods that would allow a better delineation of tumor margins intra-operatively would diminish the probability of local tumor recurrence in a sizable fraction of tumor resection procedures. This would also allow less experienced surgeons do a better job in helping their patients. The present invention provides novel compositions and methods for imaging and delineating tumor margins that may prove to be useful weapons in the ongoing struggle for improved human and animal health.

### BRIEF DESCRIPTION

In one aspect, the present invention provides a functionalized monodisperse polylysine comprising a linear monodisperse polylysine chain comprising constituent lysine monomer residues containing appended C₄-C₂₄ polyalkylene glycol groups and at least one appended fluorescent dye moiety.

In another aspect, the present invention provides a method to prepare a functionalized monodisperse polylysine comprising a linear monodisperse polylysine chain comprising constituent lysine monomer residues containing appended C₄-C₂₄ polyalkylene glycol groups and at least one appended fluorescent dye moiety.

In another aspect, the present invention provides a method to image a tumour margin, the method comprising administering to a subject a functionalized monodisperse polylysine comprising a linear monodisperse polylysine chain comprising constituent lysine monomer residues containing appended C₄-C₂₄ polyalkylene glycol groups and at least one appended fluorescent dye moiety and optically imaging the margins of a tumor within said subject.

These and other features, aspects, and advantages of the present invention may be understood more readily by reference to the following detailed description.

### BRIEF DESCRIPTION OF THE DRAWING FIGURES

FIG. 1 shows differences in tumor margin uptake of discrete polylysines compared to polydisperse polylysines having similar molecular weights according to one embodiment of the invention.

FIG. 2 shows differences in tumor margin uptake of discrete polylysines compared to polydisperse polylysines having similar chain lengths according to one embodiment of the invention.

FIG. 3 shows the effect of PEG length and structure on uptake in tumor margin and surrounding skin according to one embodiment of the invention.

FIG. 4 is a screenshot illustrating the selection of a tumor and identification of the tumor margin according to one embodiment of the invention.

FIG. 5 is a screenshot illustrating the identification of a tumor margin and display of quantitative characteristics associated with the margin according to one embodiment of the invention.

### DETAILED DESCRIPTION

In the following specification and the claims, which follow, reference will be made to a number of terms, which shall be defined to have the following meanings.

The singular forms “a”, “an” and “the” include plural referents unless the context clearly dictates otherwise.

“Optional” or “optionally” means that the subsequently described event or circumstance may or may not occur, and that the description includes instances where the event occurs and instances where it does not.

As used herein, the term “solvent” can refer to a single solvent or a mixture of solvents.

Approximating language, as used herein throughout the specification and claims, may be applied to modify any quantitative representation that could permissibly vary without resulting in a change in the basic function to which it is related. Accordingly, a value modified by a term or terms, such as “about”, is not to be limited to the precise value specified. In some instances, the approximating language may correspond to the precision of an instrument for measuring the value.

As used herein, the term “aromatic radical” refers to an array of atoms having a valence of at least one comprising at least one aromatic group. The array of atoms having a valence of at least one comprising at least one aromatic group may include heteroatoms such as nitrogen, sulfur, selenium, silicon and oxygen, or may be composed exclusively of carbon and hydrogen. As used herein, the term “aromatic radical” includes but is not limited to phenyl, pyridyl, furanyl, thienyl, naphthyl, phenylene, and biphenyl radicals. As noted, the aromatic radical contains at least one aromatic group. The aromatic group is invariably a cyclic structure having 4n+2 “delocalized” electrons where “n” is an integer equal to 1 or greater, as illustrated by phenyl groups (n=1), thienyl groups (n=1), furanyl groups (n=1), naphthyl groups (n=2), azulenyl groups (n=2), anthraceneyl groups (n=3) and the like. The aromatic radical may also include nonaromatic components. For example, a benzyl group is an aromatic radical, which comprises a phenyl ring (the aromatic group) and a methylene group (the nonaromatic component). Similarly a tetrahydronaphthyl radical is an aromatic radical comprising an aromatic group (C₆H₃) fused to a nonaromatic component —(CH₂)₄—. For convenience, the term “aromatic radical” is defined herein to encompass a wide range of functional groups such as alkyl groups, alkenyl groups, alkynyl groups, haloalkyl groups, haloaromatic groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylphenyl radical is a C₇ aromatic radical comprising a methyl group, the methyl group being a functional group which is an alkyl group. Similarly, the 2-nitrophenyl group is a C₆ aromatic radical comprising a nitro group, the nitro group being a functional group. Aromatic radicals include halogenated aromatic radicals such as 4-trifluoromethylphenyl, hexafluoroisopropylidenebis(4-phen-1-yloxy) (i.e., OPhC(CF₃)₂PhO—), 4-chloromethylphen-1-yl, 3-trifluorovinyl-2-thienyl, 3-trichloromethylphen-1-yl (i.e., 3-CCl₃Ph-), 4-(3-bromoprop-1-yl)phen-1-yl (i.e., 4-BrCH₂CH₂CH₂Ph-), and the like. Further examples of aromatic radicals include 4-allyloxyphen-1-oxy, 4-aminophen-1-yl (i.e., 4-H₂NPh-), 3-aminocarbonylphen-1-yl (i.e., NH₂COPh-), 4-benzoylphen-1-yl, dicyanomethylidenebis(4-phen-1-yloxy) (i.e., —OPhC(CN)₂PhO—), 3-methylphen-1-yl, methylenebis(4-phen-1-yloxy) (i.e., —OPhCH₂PhO—), 2-ethylphen-1-yl, phenylethenyl, 3-formyl-2-thienyl, 2-hexyl-5-furanyl, hexamethylene-1,6-bis(4-phen-1-yloxy) (i.e., —OPh(CH₂)₆PhO—), 4-hydroxymethylphen-1-yl (i.e., 4-HOCH₂Ph-), 4-mercaptomethylphen-1-yl (i.e., 4-HSCH₂Ph-), 4-methylthiophen-1-yl (i.e., 4-CH₃SPh-), 3-methoxyphen-1-yl, 2-methoxycarbonylphen-1-yloxy (e.g., methyl salicyl), 2-nitromethylphen-1-yl (i.e., 2-NO₂CH₂Ph), 3-trimethylsilylphen-1-yl, 4-t-butyldimethylsilylphen-1-yl, 4-vinylphen-1-yl, vinylidenebis(phenyl), and the like. The term “a C₃-C₁₀ aromatic radical” includes aromatic radicals containing at least three but no more than 10 carbon atoms. The aromatic radical 1-imidazolyl (C₃H₂N₂—) represents a C₃ aromatic radical. The benzyl radical (C₇H₇—) represents a C₇ aromatic radical.

As used herein the term “cycloaliphatic radical” refers to a radical having a valence of at least one, and comprising an array of atoms which is cyclic but which is not aromatic. As defined herein a “cycloaliphatic radical” does not contain an aromatic group. A “cycloaliphatic radical” may comprise one or more monocyclic components. For example, a cyclohexylmethyl group (C₆H₁₁CH₂—) is a cycloaliphatic radical, which comprises a cyclohexyl ring (the array of atoms which is cyclic but which is not aromatic) and a methylene group (the noncyclic component). The cycloaliphatic radical may include heteroatoms such as nitrogen, sulfur, selenium, silicon and oxygen, or may be composed exclusively of carbon and hydrogen. For convenience, the term “cycloaliphatic radical” is defined herein to encompass a wide range of functional groups such as alkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylcyclopent-1-yl radical is a C₆ cycloaliphatic radical comprising a methyl group, the methyl group being a functional group which is an alkyl group. Similarly, the 2-nitrocyclobut-1-yl radical is a C₄ cycloaliphatic radical comprising a nitro group, the nitro group being a functional group. A cycloaliphatic radical may comprise one or more halogen atoms which may be the same or different. Halogen atoms include, for example; fluorine, chlorine, bromine, and iodine. Cycloaliphatic radicals comprising one or more halogen atoms include 2-trifluoromethylcyclohex-1-yl, 4-bromodifluoromethylcyclooct-1-yl, 2-chlorodifluoromethylcyclohex-1-yl, hexafluoroisopropylidene-2,2-bis(cyclohex-4-yl) (i.e., —C₆H₁₀C(CF₃)₂ C₆H₁₀—), 2-chloromethylcyclohex-1-yl, 3-difluoromethylenecyclohex-1-yl, 4-trichloromethylcyclohex-1-yloxy, 4-bromodichloromethylcyclohex-1-ylthio, 2-bromoethylcyclopent-1-yl, 2-bromopropylcyclohex-1-yloxy (e.g., CH₃CHBrCH₂C₆H₁₀O—), and the like. Further examples of cycloaliphatic radicals include 4-allyloxycyclohex-1-yl, 4-aminocyclohex-1-yl (i.e., H₂C₆H₁₀—), 4-aminocarbonylcyclopent-1-yl (i.e., NH₂COC₅H₈—), 4-acetyloxycyclohex-1-yl, 2,2-dicyanoisopropylidenebis(cyclohex-4-yloxy) (i.e., —OC₆H₁₀C(CN)₂C₆H₁₀O—), 3-methylcyclohex-1-yl, methylenebis(cyclohex-4-yloxy) (i.e., —OC₆H₁₀CH₂C₆H₁₀O—), 1-ethylcyclobut-1-yl, cyclopropylethenyl, 3-formyl-2-terahydrofuranyl, 2-hexyl-5-tetrahydrofuranyl, hexamethylene-1,6-bis(cyclohex-4-yloxy) (i.e., —OC₆H₁₀(CH₂)₆C₆H₁₀O—), 4-hydroxymethylcyclohex-1-yl (i.e., 4-HOCH₂C₆H₁₀—), 4-mercaptomethylcyclohex-1-yl (i.e., 4-HSCH₂C₆H₁₀—), 4-methylthiocyclohex-1-yl (i.e., 4-CH₃SC₆H₁₀—), 4-methoxycyclohex-1-yl, 2-methoxycarbonylcyclohex-1-yloxy (2-CH₃OCOC₆H₁₀O—), 4-nitromethylcyclohex-1-yl (i.e., NO₂CH₂C₆H₁₀—), 3-trimethylsilylcyclohex-1-yl, 2-t-butyldimethylsilylcyclopent-1-yl, 4-trimethoxysilylethylcyclohex-1-yl (e.g., (CH₃O)₃SiCH₂CH₂C₆H₁₀—), 4-vinylcyclohexen-1-yl, vinylidenebis(cyclohexyl), and the like. The term “a C₃-C₁₀ cycloaliphatic radical” includes cycloaliphatic radicals containing at least three but no more than 10 carbon atoms. The cycloaliphatic radical 2-tetrahydrofuranyl (C₄H₇O—) represents a C₄ cycloaliphatic radical. The cyclohexylmethyl radical (C₆H₁₁CH₂—) represents a C₇ cycloaliphatic radical.

As used herein the term “aliphatic radical” refers to an organic radical having a valence of at least one consisting of a linear or branched array of atoms, which is not cyclic. Aliphatic radicals are defined to comprise at least one carbon atom. The array of atoms comprising the aliphatic radical may include heteroatoms such as nitrogen, sulfur, silicon, selenium and oxygen or may be composed exclusively of carbon and hydrogen. For convenience, the term “aliphatic radical” is defined herein to encompass, as part of the “linear or branched array of atoms which is not cyclic” a wide range of functional groups such as alkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylpent-1-yl radical is a C₆ aliphatic radical comprising a methyl group, the methyl group being a functional group which is an alkyl group. Similarly, the 4-nitrobut-1-yl group is a C₄ aliphatic radical comprising a nitro group, the nitro group being a functional group. An aliphatic radical may be a haloalkyl group which comprises one or more halogen atoms which may be the same or different. Halogen atoms include, for example; fluorine, chlorine, bromine, and iodine. Aliphatic radicals comprising one or more halogen atoms include the alkyl halides trifluoromethyl, bromodifluoromethyl, chlorodifluoromethyl, hexafluoroisopropylidene, chloromethyl, difluorovinylidene, trichloromethyl, bromodichloromethyl, bromoethyl, 2-bromotrimethylene (e.g., —CH₂CHBrCH₂—), and the like. Further examples of aliphatic radicals include allyl, aminocarbonyl (i.e., —CONH₂), carbonyl, 2,2-dicyanoisopropylidene (i.e., —CH₂C(CN)₂CH₂—), methyl (i.e., —CH₃), methylene (i.e., —CH₂—), ethyl, ethylene, formyl (i.e., —CHO), hexyl, hexamethylene, hydroxymethyl (i.e., —CH₂OH), mercaptomethyl (i.e., —CH₂SH), methylthio (i.e., —SCH₃), methylthiomethyl (i.e., —CH₂SCH₃), methoxy, methoxycarbonyl (i.e., CH₃OCO—), nitromethyl (i.e., —CH₂NO₂), thiocarbonyl, trimethylsilyl (i.e., (CH₃)₃Si—), t-butyldimethylsilyl, 3-trimethyoxysilylpropyl (i.e., (CH₃O)₃SiCH₂CH₂CH₂—), vinyl, vinylidene, and the like. By way of further example, a C₁-C₁₀ aliphatic radical contains at least one but no more than 10 carbon atoms. A methyl group (i.e., CH₃—) is an example of a C₁ aliphatic radical. A decyl group (i.e., CH₃(CH₂)₉—) is an example of a C₁₀ aliphatic radical.

As used herein the term “appended” is defined as “attached to via a covalent bond”.

As noted, in one embodiment, the present invention provides a functionalized monodisperse polylysine composition comprising a linear monodisperse polylysine chain. As used herein, the term “monodisperse polylysine” means that the polylysine backbone is substantially a single molecular entity. The linear monodisperse polylysine chain includes constituent lysine residues having structure I

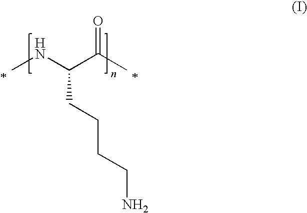

In one embodiment, the polylysine is a oligomeric or polymeric molecule. In one embodiment, the polylysine is a monodisperse polylysine. In one embodiment, the monodisperse polylysine is a monodisperse homopolymer having in a range from about 25 to about 200 residues.

In one embodiment, the polylysine is poly L-lysine, poly D-lysine, or a combination thereof. In one embodiment, the polylysine is a homopolymer. In another embodiment, the polylysine is a copolymer for example a copolymer such as a D-Tyr-D-Lys copolymer. In one embodiment, the lysine residues are present in the backbone, the ε-amino “groups” on the side chains of the lysine residues can serve as convenient reactive groups for covalent linkage of at least one fluorescent dye and other modifying groups such as polyethylene glycol. For example, a carboxyl group (or an activated ester) on the fluorescent dye can be used to form an amide linkage with a primary amine such as the ε-amino group of the lysyl side chain on polylysine.

The monodisperse polylysine serves as biocompatible template which can be readily modified and to which can be attached at least one fluorescent dye. In one embodiment, the fluorescent dye is attached to a terminal amine group of the monodisperse polylysine. In another embodiment, the fluorescent dye can be attached to one or more of the ε-amino groups of the monodisperse polylysine. In one embodiment, the polylysine design will depend on considerations such as biocompatibility (e.g., toxicity and immunogenicity), serum half-life, functional groups (for conjugating chromophores, spacers, and protective groups), and cost. In one embodiment, the polylysine can include polypeptides (polyamino acids), where a majority of the amino acid side chains are functionalized with one or more dyes and polyalkylene glycol groups.

In one embodiment, the constituent lysine monomer residues are greater than 90% functionalized. In another embodiment, the constituent lysine monomer residues are greater than 95% functionalized.

In one embodiment, the monodisperse polylysine has a molecular weight in a range from about 5,000 Daltons to about 500,000 Daltons as determined by gel filtration chromatography. In another embodiment, the monodisperse polylysine has an average molecular weight in a range from about 10,000 Daltons to about 300,000 Daltons, as determined by gel filtration chromatography. In one embodiment, the monodisperse polylysine has a molecular weight in the range from about 15000 Daltons to about 100000 Daltons as determined by gel filtration chromatography. In another embodiment, the monodisperse polylysine has an average molecular weight at least about 30k Daltons as determined by gel filtration chromatography.

The compositions provided by the present invention comprise a functionalized monodisperse polylysine to which have been appended C₄-C₂₄ polyalkylene glycol groups. In one embodiment, the appended polyalkylene glycol groups are selected from the group consisting of C₄-C₂₄ polyethylene and C₆-C₃₆ polypropylene glycol groups. In yet another embodiment, the appended polyalkylene glycol groups are C₄-C₂₄ polyethylene glycol groups only. In another embodiment, the appended polyalkylene glycol groups are C₄-C₁₂ polyethylene glycol groups. In one embodiment, appended polyalkylene glycol groups are linear. In an alternate embodiment, the appended polyalkylene glycol groups are branched. In one embodiment, the functionalized monodisperse polylysine composition provided by the present invention comprises at least one appended polyalkylene glycol group having structure (II).

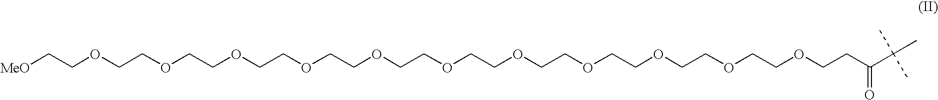

In one embodiment, the appended polyalkylene glycol groups have a molecular weight in a range from about 130 Daltons to about 1500 Daltons. In another embodiment, the appended polyalkylene glycol groups have an average molecular weight in a range from about 200 Daltons to about 800 Daltons.

In one embodiment, the appended polyalkylene glycol group is a homopolymer. In another embodiment, appended polyalkylene glycol group is a block copolymer comprising, for example, polyethylene glycol and polypropylene glycol structural units. In one embodiment, the appended polyalkylene glycol group is a functionalized polyalkylene glycol group, such as for example methoxypolyethylene glycol (MPEG), methoxypolypropylene glycol, polyethylene glycol-diacid, polyethylene glycol monoamine, methoxypolyethylene glycol monoamine, methoxypolyethylene glycol hydrazide, and methoxypolyethylene glycol imidazolide.

The functionalized monodisperse polylysines provided by the present invention comprise at least one appended fluorescent dye moiety.

In one embodiment, the at least one appended fluorescent dye moiety is selected from the following general classes of compounds (III)-(VII) (and their pharmaceutically acceptable salts).

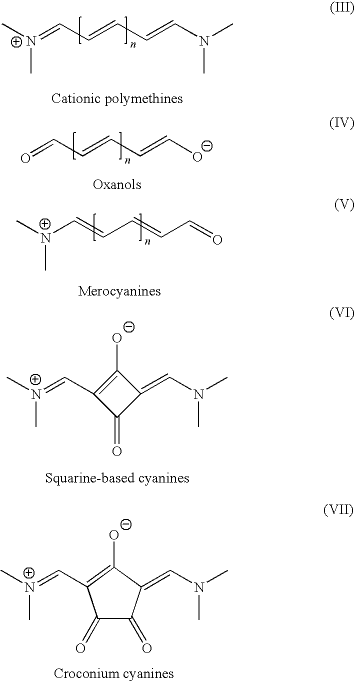

In one embodiment, the at least one appended fluorescent dye moiety has a structure VIII

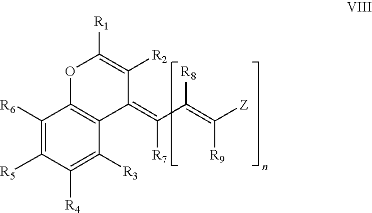

wherein Z is a substituted or unsubstituted benzoxazol group, a substituted or unsubstituted benzothiazol group, a substituted or unsubstituted 2,3,3-trimethylindolenine group, a substituted or unsubstituted 2,3,3-trimethyl-4,5-benzo-3H-indolenine group, a substituted or unsubstituted 3- or 4-picoline group, a substituted or unsubstitute lepidine group, a substituted or unsubstituted chinaldine group, or a substituted or unsubstituted 9-methylacridine group. Or Z may have be a group having one of structures IX, X or XI.

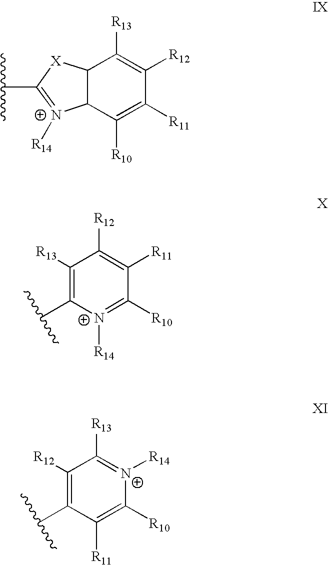

In reference to the foregoing structures VIII-XI, the moiety “X” is an element selected from the group consisting of O, S, Se; or the moiety “X” may be N-alkyl group or a C(alkyl)₂ group; “n” is 1, 2 or 3; R₁-R₁₄ are the same or different and can be hydrogen, a C₁-C₃₀ aliphatic radical, a C₃-C₃₀ cycloaliphatic radical, a C₂-C₃₀ aromatic radical, a hydroxyl group, an alkoxy group, or a halogen. In one embodiment, R₁-R₁₄ is a cyclical amine function and/or two fragments in ortho position to each other, for example R₁₀ and R₁₁, can together form another aromatic ring; at least one of the substituents R₁-R₁₄ can be a solubilizing or ionizable or ionized substituent, such as a polyethyleneglycol, cyclodextrin, sugar, SO₃⁻, PO₃²⁻, COO⁻, or NR₃⁺, which determines the hydrophilic properties of these dyes. In some embodiments, the solubilizing or ionizable or ionized substituent is bound to the dye by means of a spacer group. In some embodiments, at least one of the substituents R₁-R₁₄ can be a reactive group, which facilitates appending the dye to the polylysine or the polyethylene glycol group. In other embodiments, R₁ is a substituents, which has a quaternary C-atom in alpha-position relative to the pyran ring, e.g., t-butyl and adamantyl. Non limiting examples the fluorescent dyes include compounds having structures XII-XVII

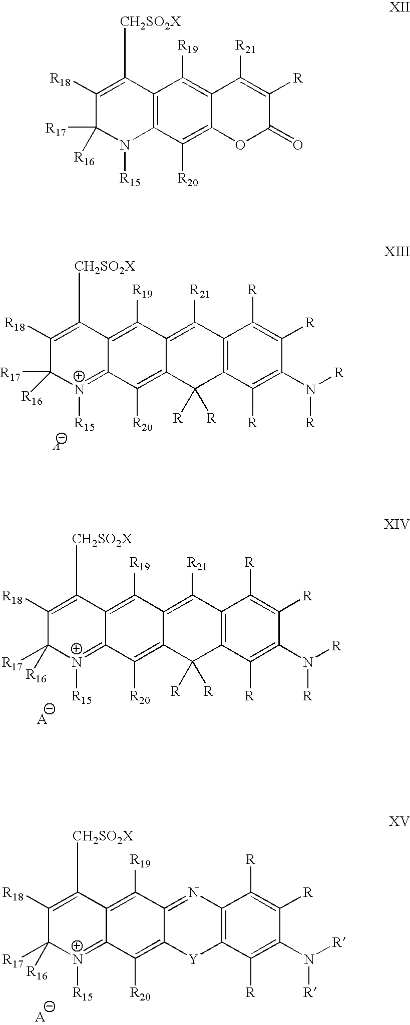

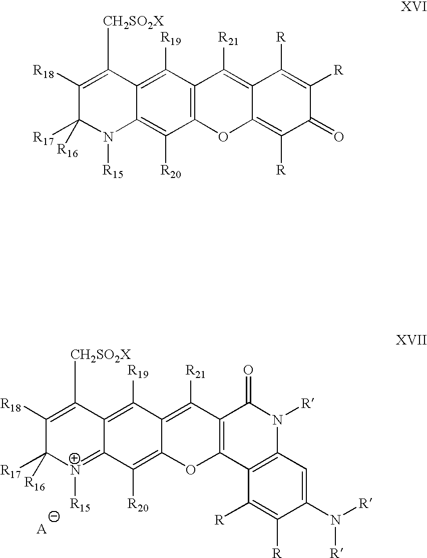

wherein R₁₅ denotes hydrogen, a C₁-C₂₀ aliphatic radical, a C₃-C₂₀ cycloaliphatic radical, or a C₃-C₂₀ aromatic radical; R₁₆, R₁₇, R₁₈, R₁₉, and R₂₀ on each occurrence and independently of one another denote hydrogen, halogen, a hydroxy, amino, sulfo, carboxy or aldehyde group or a C₁-C₂₀ aliphatic radical, a C₃-C₂₀ cycloaliphatic radical, a C₃-C₂₀ aromatic radical, or the residues R₁₅ and R₂₀ together form a ring system; R on each occurrence can be the same or different and is defined as for R₁₅, R₁₆, R₁₇, R₁₈, R₁₉ and R₂₀; R′ on each occurrence and independently of one another denotes hydrogen, a C₁-C₂₀ aliphatic radical, a C₃-C₂₀ cycloaliphatic radical, or a C₃-C₂₀ aromatic radical, or the residues R and R′ together form a ring system which can contain one or more double bonds; R₂₁ on each occurrence and independently of one another denotes hydrogen, a C₁-C₂₀ aliphatic radical, a C₃-C₂₀ cycloaliphatic radical, or a C₃-C₂₀ aromatic radical; X denotes OH, halogen, —O—R₂₂, —S—R₂₃ or —NR₂₄R₂₅ where R₂₂, R₂₃, R₂₄, and R₂₅ each independently of one another denote hydrogen a C₁-C₂₀ aliphatic radical, a C₃-C₂₀ cycloaliphatic radical, or a C₃-C₂₀ aromatic radical; and Y in structure XV denotes O, S or C(R)₂. In one embodiment, R₂₁ is a hydrogen, a carboxyphenyl, a perfluoroalky, a pyridyl or a carboxypyridyl.

In one embodiment, the fluorescent dye has a structure XVIII:

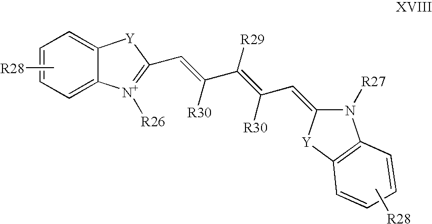

wherein, as valence and stability permit, Y represents C(R₃₀)₂, S, Se, O, or NR₃₁; R₃₀ represents H or C₁-C₂₀ aliphatic radical, a C₃-C₃₀ cycloaliphatic radical, or two occurrences of R₃₀, taken together, form a ring together with the carbon atoms through which they are connected; R₂₆ and R₂₇ represent, independently, C₁-C₂₀ aliphatic radical, a C₃-C₃₀ cycloaliphatic radical, a C₃-C₃₀ aromatic radical optionally substituted by sulfate, phosphate, sulfonate, phosphonate, halogen, hydroxyl, amino, cyano, nitro, carboxylic acid, amide, etc., or a pharmaceutically acceptable salt thereof; R₂₈ represents, independently for each occurrence, one or more substituents to the ring to which it is attached, such as a fused ring (e.g., a benzo ring), sulfate, phosphate, sulfonate, phosphonate, halogen, hydroxyl, amino, cyano, nitro, carboxylic acid, amide, a C₁-C₂₀ aliphatic radical, a C₃-C₃₀ cycloaliphatic radical, a C₃-C₃₀ aromatic radical, or a pharmaceutically acceptable salt thereof; R₂₉ represents H, halogen, or a substituted or unsubstituted ether or thioether of phenol or thiophenol. Dyes representative of this formula include indocyanine green

Non-limiting examples of the at least one fluoroscent dye moiety is Cy5.5, Cy5, and Cy7, (Amersham, Arlington Hts., IL); IRDye78, IRDye80, IRDye38, IRDye40, IRDye41, IRDye700, IRDye800 (LI-COR, Lincoln, Nebr.); NIR-1, IC5-OSu, (Dejindo, Kumamoto, Japan); LaJolla Blue (Diatron, Miami, Fla.); Alexaflour 660, Alexflour 680 (Molecular Probes, Eugene, Oreg.), FAR-Blue, FAR-Green One, FAR-Green Two (Innosense, Giacosa, Italy); ADS 790-NS, ADS 821-NS (American Dye Source, Montreal, Canada); indocyanine green (ICG) and analogs thereof, indotricarbocyanine (ITC; WO 98/47538); chelated lanthanide compounds that display near infrared optical properties, and fluorescent quantum dots (zinc sulfide-capped cadmium selenide nanocrystals) (e.g., QuantumDot Corporation; www.qdots.com) and chelated lanthanide compounds.

In another embodiment, the at least one appended fluorescent dye moiety is a phenothiazines such as methylene blue, a cyanine dye (for example Cy5, Cy5.5, Cy7, Cy7.5, ICG, SIDAG, Alexa 647 Alexa 680, Alexa 750, IR800, Dylight 649, Omocianine), a xanthene dye (for example fluoresceins, rhodamines), a large Stoke shift cyanine dye (US20080206886A), a phenoxazine dye (for example resorufin, Atto dyes), a benzopyrelium dye (for example Dy650, Dy681, Dy752 etc.), a merocyanine dye, an acridinone dye (for example DDAO) or a styryl dye.

In yet another embodiment, the cyanine dye is at least one selected from a streptocyanine dye, a hemicyanine dye or a closed chain cyanine. In one embodiment, the at least one fluorescent dye has a structure XIX

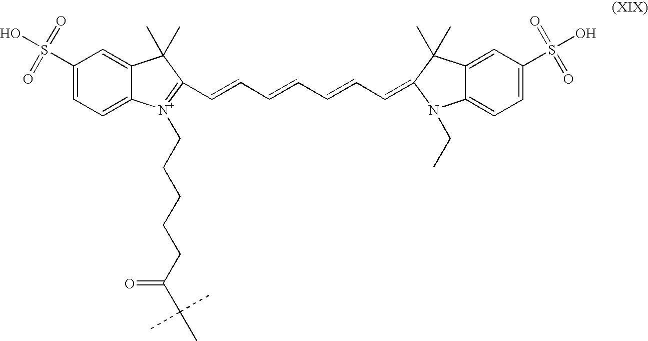

In one embodiment, the “pharmaceutically acceptable salts” of the dyes mentioned above can be used. As used herein, “pharmaceutically acceptable salts” refers to derivatives of the disclosed compounds wherein the parent compound is modified by making the acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Conventional non-toxic salts also include those derived from bases such ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

In one embodiment, the at least one fluoroscent dye moiety can be covalently linked to the polylysine moiety including the spacers, using any suitable reactive group on the at least one fluoroscent dye moiety and a compatible functional group on the polylysine moiety or spacer.

Not all cyanine and related dyes are luminescent. However, the dyes of this invention include those of the cyanine and related dyes, which are luminescent. In one embodiment, the dyes are relatively photostable and many are soluble in the reaction solution. In one embodiment, the dyes themselves, but more particularly when conjugated to the monodisperse polylysine, have molar extinction coefficients (ε) of at least 25,000 but preferably at least 50,000 per mole centimeter. The extinction coefficient is a measure of the capability of the molecules to absorb light. In one embodiment, the at least one appended fluorescent dye moiety has an excitation and emission wavelength in a range from about 400 nm to about 1300 nm. In another embodiment, the at least one appended fluorescent dye moiety has an excitation and emission wavelength in a range from about 600 nm to about 900 nm. In yet another embodiment, the at least one appended fluorescent dye moiety has an excitation and emission wavelength in a range from about 650 nm to about 800 nm. In one embodiment, the number of fluorescent dye moieties is about 1 per polylysine chain.

In one embodiment, the polyethylene glycol and the at least one fluorescent dye moiety contains at least one, group that can react covalently to an amine, hydroxy, aldehyde or sulfhydryl group on the polylysine. Non-limiting examples of such groups include isothiocyanate, isocyanate, hydroxysuccinimide ester, hydroxysulfosuccinimide ester group, halogenacetyl groups, ester groups, carboxy groups and the like.

In one embodiment, the functionalized monodisperse polylysine further includes pharmaceutically acceptable carriers, adjuvants, vehicles, include, but are not limited to, ion exchangers, alumina, aluminum stearate, lecithin, serum proteins such as albumin, buffer substances such as phosphate, glycine, sorbic acid, potassium sorbate, TRIS (tris(hydroxymethyl)amino methane), partial glyceride mixtures of fatty acids, water, salts or electrolytes, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polypropylene block polymers, sugars such as glucose, and suitable cryoprotectants.

In one embodiment, monodisperse polylysine backbone can be prepared by chemical ligation method. In one embodiment, the chemical ligation of two short monodisperse polylysine chains can be used where each short monodisperse polylysine chain can be synthesized on a peptide synthesizer and ligated to another chain prior to functionalization and conjugation with a dye. In another embodiment, the short monodisperse polylysine chain can be synthesized on a peptide synthesizer and ligated to another chain after the chains have been functionalized.

In one embodiment, the functionalized monodisperse polylysine of the invention may be used for visualization of tissue without pathological alterations, systemic diseases, tumors, blood vessels, atherosclerotic plaques, perfusion and diffusion. In one embodiment, the compositions of the present invention may be used in combination with other imaging compositions and methods, for example, the methods of the present invention may be used in combination with imaging modalities such as CT, PET/SPECT or MRI, and probes used in these methods can contain components, such as iodine, gadolinium atoms or radioactive isotopes, which change imaging characteristics of tissues when imaged using CT, PET, SPECT, or MR. In another embodiment, the functionalized monodisperse polylysine composition of the present invention includes at least one fluorescent dye moiety chemically linked to the polyethylene glycol group. In one embodiment, the functionalized monodisperse polylysine can be combined with therapeutic methods. For example, if the probes of the present invention detect a tumor, an immediate anti-tumor therapy can be employed.

### EXAMPLES

The following examples illustrate methods and embodiments in accordance with the invention, and as such do not limit the claims. H-Val-H NovaSyn TG resin and N-Boc-protected aminoxyacetic acid (wherein “Boc”=tert-butyl carbamate) were purchased from Novabiochem (San Diego, Calif.), N-α-Fmoc-(N-ε-Boc)-lysine, trifluoroacetic acid (herein also known as “TFA”) (wherein Fmoc=9-fluorenylmethyl carbamate), ortho-benzotriazole-N,N,N′,N′-tetramethyluronium hexafluorophosphate (herein also known as “HBTU”), and 4-(2′,4′-dimethoxyphenyl-Fmoc-aminomethyl)-phenoxy resin (rink amide resin LS, 100-200 mesh, 1% divinylbenzene (herein also known as “DVB”), 0.2 mmol/g) were purchased from Advanced Chemtech (Louisville, Ky.). Dimethylformamide (herein also known as “DMF”) and methylene chloride were from Fisher Scientific (Fair Lawn, N.J.). 20% piperidine in DMF and N-methylmorpholine (herein also known as “NMM”) in DMF were from Protein Technologies Inc. (Tucson, Ariz.). Pyridine, acetic anhydride, acetic acid, and anhydrous ether were purchased from J. T. Baker (Phillipsburg, N.J.) triisopropylsilane (herein also known as “TIPS”), was purchased from Aldrich (Milwaukee, Wis.).

### General Procedure for Synthesis of Monodisperse Polylysine Using Solid Phase Method

Monodisperse polylysine was synthesized using standard solid phase techniques with N-α-Fmoc-protected amino acids using 0.2 mmol/g substitution Rink Amide Resin LS or 0.23 mmol/g substitution H-Val-H methyloxazolidine NovaSyn® TG resin (Merck Chemials, Darmstadt, Germany) in a 100 μmole scale. The polylysines were synthesized either using a Symphony peptide synthesizer (Protein Technologies Inc, Arizona, USA) or a Prelude peptide synthesizer (Protein Technologies Inc, Arizona, USA). The resin was swelled for one hour in methylene chloride, and was subsequently washed with DMF for about 30 minutes when the methylene chloride was exchanged. Each coupling reaction was carried out at room temperature with HBTU as coupling reagent and NMM as the base. For each step, the coupling agent and the amino acid were each delivered at a scale of five equivalents relative to the estimated resin capacity. Double couplings were carried out for most residues except for the residues 2-5. The coupling time was about 30 minutes for a single coupling and typically about 2×30 minutes for a double coupling. The reactions did not perturb the side-chains of the amino acids, which were protected with an acid labile Boc group.

Following each coupling reaction, the N-terminal Fmoc-protected amine was deprotected by applying 20% piperidine in DMF at room temperature for approximately 15 minutes.

### Preparation of Cy5-PL₅₀ (Cy5-K₅₀-NH₂)

After the final amino acid addition, Fmoc group was removed while the polylysine was still on the solid support and solid support was washed with DMF about six times, DCM about six times and dried for about 30 minutes by passing nitrogen through the reaction vessel. The solid support was then dried in vacuum overnight.

The dye was attached to the polylysine while the polylysine was on the solid support and all the amino acid side chains were protected limiting the dye attachment to the amine-terminus, thereby, preventing formation of multiple species. A portion of the solid support (about ⅓^{rd} of total) was placed back on the peptide synthesizer and was swollen in dichloromethane (DCM) for 30 minutes and washed three times each with DCM and DMF followed by suspending the treated solid support in 4 ml of anhydrous DMF. To the suspension about 40 microliteres of N-methylmorpholine was added followed by the addition of a solution of the dye, Cy5—NHS ester (23.5 mg, 97% active ester, 1.2 equivalent) in about 1 ml of anhydrous DMF. The dye container was rinsed with about 1 ml of DMF and this solution was also added to the reaction mixture. The reaction mixture was allowed to stand overnight with agitation about every 30 seconds by bubbling nitrogen through the suspension. The dye solution was then allowed to drain and the support was repeatedly washed with DMF (about 9 times) and then DCM (about 6 times) before drying. The support was then dried by passing nitrogen for about 30 minutes. Following this, the polylysine was cleaved from the support and the epsilon amino groups were deprotected by agitating the support with 2 ml of a mixture of TFA:Water:TIPS (triisopropylsilane) in a ratio of about 95:2.5:2.5 respectively, for about 4 hours. The solution was filtered and the residue was washed with water until the wash was colorless.

The filtrate and the washes were concentrated and the residue was purified on an AKTA purifier using Xterra MS C18 30×100 mm preparation column with 0.1% TFA in water as buffer A and 0.1% TFA in acetonitrile as buffer B. A gradient method, 0-10% B in two column volumes (CV), 10-20% B in 25CV and 20-100% B in 1CV with a flow rate of 25 ml/min was used to elute the column. Fractions were checked by analytical HPLC. The pure fractions (HPLC fractions (38-48) from AKTA purification) were combined, concentrated to dryness and co-evaporated with water (about 3 times). The residue was dissolved in water (1.3 ml) and the concentration was calculated by UV/VIS analysis method. Any unlabeled polylysine, which has a very different mobility on reverse phase HPLC compared to the labeled polylysine, was removed. A yield of about 5% of the product Cy5-PL₅₀, was obtained with an average molecular weight 7063.4 (Calc 7062.7).

### Example 1

### Preparation of Cy5-PL₅₀-EG₁₂ (Cy5-(K-EG₁₂)₅₀)

To the Cy5-PL50 solution prepared by the procedure above, 1 molar sodium bicarbonate and distilled water was added such that the final concentration of 10 mg/ml in 0.1M sodium bicarbonate at a pH of about 8.6 was obtained. The pH was adjusted by addition of NaOH. A solution of about 3.13 equivalents (per lysine unit) of m-dPEG™₁₂ NHS ester was prepared in anhydrous dimethylsulfoxide (DMSO, about 1.75M) and was added to the Cy5-PL₅₀ solution with vigorous stirring. The stirring was carried out at room temperature overnight and diluted with water to reduce DMSO content to less than about 5%. The reaction mixture was filtered using an Amicon 5K MW cutoff filter on a Bechman-Coulter Allegra bench-top centrifuge at about 4° C. and about 3500 rotations per minute (rpm) for about 30 minutes. The residue was repeatedly washed with water to remove the low molecular weight species. The removal of low molecular weight species was observed periodically in the residue by GPC chromatography. The washed residue was then dissolved in water and the concentration was measured using UV-visible spectroscopy by using the absorbance of the dye. A yield of about 78% was obtained with the average molecular weight as measured using MALDI analysis of about 34000 (calculated for a 100% pegylated product=35,604) indicating that a majority of polymer units were fully functionalized.

The above procedure was employed to prepare Cy5-PL₅₀-EG₄, Cy5-PL₅₀-EG₄(EG₁₂)₃, Cy5-PL₅₀-EG₁₆

### Preparation of Cy5-PL₅₀-Val-CHO (Cy5-K₅₀-V-CHO)

The compound Cy5-PL50-Val-CHO was prepared on 100 umole scale on a H-Val-H NovaSyn® TG resin (for peptides with aldehyde functionality on the carboxy terminal) solid support, employing a method similar to the method described above for Cy5-PL₅₀. After final amino acid addition, Fmoc group was removed while the polylysine was still on the solid support and the solid support was washed with DMF (about six times), DCM (about six times) and dried for about 30 minutes by passing nitrogen through the reaction vessel. The solid support was then dried in vacuum overnight.

The dye was attached to the polylysine while the polylysine was on solid support and all amino acid side chains were protected limiting the dye attachment to the amine-terminus, thereby, preventing formation of multiple species. A portion of the solid support (about ½ of total) was placed back on the peptide synthesizer and was swollen in dichloromethane (DCM) for 30 minutes and washed three times each with DCM and DMF followed by suspending the treated solid support in 5 ml of anhydrous DMF. To the suspension about 45 microliteres of N-methylmorpholine was added followed by addition of a solution of the dye, Cy5-NHS ester (28.7 mg, 97% active ester) in about 1 ml of anhydrous DMF. The dye container was rinsed with about 1 ml of DMF and this solution was also added to the reaction mixture. The reaction mixture was allowed to stand overnight with agitation every 30 seconds by bubbling nitrogen through the suspension. The Dye solution was then allowed to drain and the support was repeatedly washed with DMF (about 9 times) and then DCM (about 6 times) before drying. The support was then dried by passing nitrogen for about 30 minutes.

Following this, the epsilon amino groups were deprotected in 100% TFA (2.5 ml) for about 3 hours prior to cleaving the polylysine from the support. The solution was filtered and the residue was washed with methanol. After filtration was completed the residue was taken in about 5 ml of water and stirred for 1 hour. The aqueous solution (blue color) was filtered and the residue was washed with water and methanol. The filtrate and the washes were concentrated and the residue was purified on an AKTA purifier using Xterra MS C18 30×100 mm preparative column with 0.1% TFA in water as buffer A and 0.1% TFA in acetonitrile as buffer B. A gradient of 0-20% B in 25CV, 20-30% B in 6.25CV, 30-100% B in 1CV and a flow rate of 25 ml/min were used for column elution. Buffers A & B were as described above. An average molecular weight of 7148, monoisotopic MW 7144, by electrospray MS analysis (Calc 7146) was obtained.

### Preparation of Aminoxyacetyl-PL₄₉ (NH₂O—CH₂—C(O)—K₄₉)

The Aminoxyacetyl-PL₄₉ was prepared using the general procedure for synthesis of polylysine using solid phase method mentioned above except that the last coupling was performed with di-tBOC-aminoxyacetic acid as an amino acid substitute. For the aminoxyacetyl-modified polylysine synthesis, the last synthesis cycle did not contain the Fmoc deprotection step. After the addition of the last residue, the resin, still on the peptide synthesizer, was rinsed thoroughly with DMF and methylene chloride before being dried under a stream of nitrogen for 30 minutes. The polylysine was cleaved from the support and the aminoxy group as well as the epsilon amino groups were deprotected by agitating the support with 2 ml of a mixture of TFA:Water:TIPS (triisopropylsilane) in a ratio of about 95:2.5:2.5 respectively, for about 4 hours. The solution was filtered and residue was washed with water until wash became colorless. The filtrate and the washes were concentrated and the residue was purified on an AKTA purifier using Xterra MS C18 30×100 mm preparative scale column with 0.1% TFA in water as buffer A and 0.1% TFA in acetonitrile as buffer B. A gradient of 0-9% B in 12.5CV, 9% B for 6.25CV, 9-100% B in 6.25CV and a flow rate of 25 ml/min were used to elute the column. Buffers A and B were as above. The product Aminoxyacetyl-PL49 with an average molecular weight of 6368, by an electrospray MS method (Calc 6368) was obtained.

### Preparation of Cy5-“PL₁₀₀” (Cy5-K₅₀-V-C═N—OCH₂C(O)K₄₉—NH₂)

The compounds Cy5-PL₅₀-Val-CHO (2.4 micromoles) and Aminoxyacetyl-PL₄₉ (about 7.5 equivalents) prepared by the procedure mentioned above were each dissolved in 1 ml of 50 mM ammonium acetate (the pH measured after dissolving the compounds was about 4.6). The solution of the compounds in ammonium acetate was mixed and heated at about 50° C. with stirring conditions for about 90 minutes. The reaction mixture was allowed to cool and was purified on an AKTA purifier using Xterra MS C18 30×100 mm preparative scale column with 0.1% TFA in water as buffer A and 0.1% TFA in acetonitrile as buffer B. The gradient used was 0-10% B in 2.5CV, 10-20% B in 37.5CV and 30-100% B in 1CV. The flow rate was about 25 ml/min. The fractions 47-57 were pure by analytical HPLC and were combined. The solution was concentrated to dryness and coevaporated with water three times. A yield of about 51% was obtained with the average molecular weight as measured using MALDI analysis of about 31501.7 (calc. monoisotopic mass 31,496).

### Example 2

### Preparation of Cy5-“PL₁₀₀”-EG₁₂ (Cy5-(K-EG₁₂)₅₀-(V-EG₁₂)—C═N—OCH₂(CO)(K-EG₁₂)₄₉—NH₂)

About 7.46 mg of Cy5-“PL₁₀₀” (Cy5-K₅₀-V-C═N—OCH₂(CO)K₄₉—NH₂) prepared using the procedure given above was dissolved in a 0.1M sodium bicarbonate (10 mg/ml) at pH 8.6. To the Cy5-“PL₁₀₀” solution was added a solution of m-dPEG™₁₂ NHS (3.13 equivalent per lysine unit) in 94 microliters of anhydrous DMSO with vigorous stirring. The reaction mixture was stirred at room temperature overnight in dark. Following this, the reaction mixture was diluted with water to reduce DMSO content to less than about 5% and filtered on an Amicon 5K MW cutoff filter as described above. The residue was washed until no more of the low molecular weight species were present. The product Cy5-“PL₁₀₀”-EG₁₂ ((Cy5-(K-EG₁₂)₅₀-(V-EG₁₂)—C═N—OCH₂(CO)(K-EG₁₂)₄₉—NH₂)) having an average molecular weight of 65,670.8, by MS analysis (by MALDI, calc. for 100% pegylation 69,959) was obtained.

A sample of Cy5-“PL₁₀₀”-EG₈ was prepared using the above-mentioned procedure.

### Comparative Example

### Preparation of PL₁₂₂-EG₁₂

A solution of polydisperse polylysine.HBr (97.5 mg at a conc. of 10 mg/ml, degree of polymerization (DP) is about 122 by viscosity from Sigma-Aldrich) was prepared in a 0.1M sodium bicarbonate at pH 8.8. A solution of m-dPEG™₁₂ NHS (3.13 eq per lysine unit) in anhydrous DMSO (1.22 ml/1000 mg) was added to the above polylysine solution with vigorous stirring. The stirring was carried out at room temperature overnight. After the stipulated time, the reaction mixture was diluted with water to reduce the DMSO content to less than about 5% and was filtered on Allegra centrifuge at about ° C. and about 3500 rotations per minute (rpm) for about 30 minutes. The residue was repeatedly washed with water till no significant amount of low molecular weight species were left, as determined by gel permeation chromatography (GPC). The gel permeation chromatography analyses were performed on Agilent Zorbax GF-250, 4.6×250 mm column, particle size 4 μm using 1× PBS as elution buffer at 0.5 ml/min.

### Comparative Example 1

### Synthesis of polydisperse Cy5-PL₁₂₂-EG₁₂ (Cy5-(K-EG₁₂)₁₂₂

One half of the above solution was reformulated in sodium bicarbonate to give a 4.88 ml 0.1M sodium bicarbonate solution. The dye Cy5-NHS was dissolved in anhydrous DMSO. After spectrophotometric concentration measurement by UV/VIS analysis, 71.6 microliters of 25.1 microgram per microliter of the Cy5-NHS solution (about 1.2 equivalent/polymer molecule) was added to the PL₁₂₂-EG₁₂ solution under vigorous stirring condition. The mixture was stirred at room temperature in dark overnight. After the stipulated time the mixture was filtered and washed on a Amicon 30K MW cutoff filter on a Bechman-Coulter Allegra bench-top centrifuge at about 4° C. and about 3500 rotations per minute (rpm) for about 30 minutes. The purity of the Sample was checked by gel permeation chromatography (GPC).

The subject such as a Fischer rat with angiogenic tissues, such as MatBIII rat breast adenocarcinoma tumors injected orthotopically in the mammary fat pad, was administered with a compound of Example 1 at a dose of about 125 nmol of dye/kg body weight of a subject. The subject was allowed to metabolize for different times such as 3, 6 or 24 hours. The subject was surgically opened to expose the likely tumor location in the mammary fat pad. An infrared (IR) and color camera with light emitting diodes (LEDs) were used to take the fluorescent images of the tumor margin uptake of the dye-labeled agents.

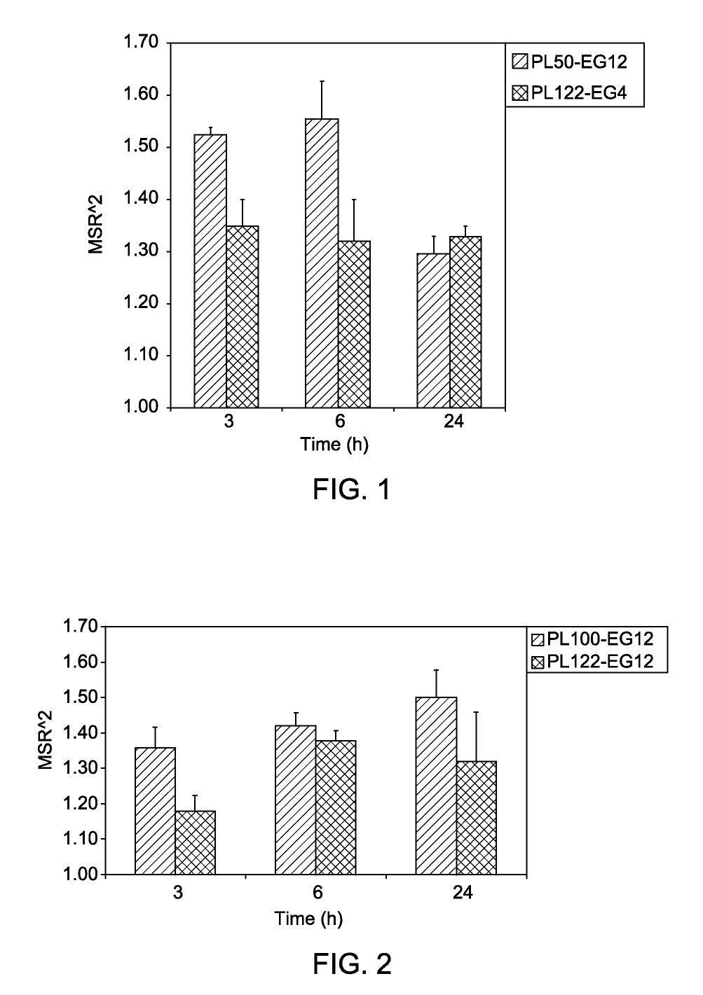

FIG. 1 shows the differences in tumor margin uptake of discrete polylysines compared to similar molecular weights polydisperse polylysines and FIG. 2 the differences in tumor margin uptake of discrete polylysines compared or similar length of the polydisperse polylysines. In FIG. 1 and FIG. 2 the Y-axis represents squared ratio of intensities in margin to surrounding skin or squared margin to surrounding skin ratio (MSR̂2). These ratios were determined using a software described below. The X-axis in FIG. 1 and FIG. 2 represents samples taken at different time points (i.e. 3, 6 & 24 h).

Method to determine the squared ratio of intensities in margin to surrounding skin or squared margin to surrounding skin ratio (MSR̂2): (U.S. patent application Ser. No. 12/259,944 incorporated herein by reference)

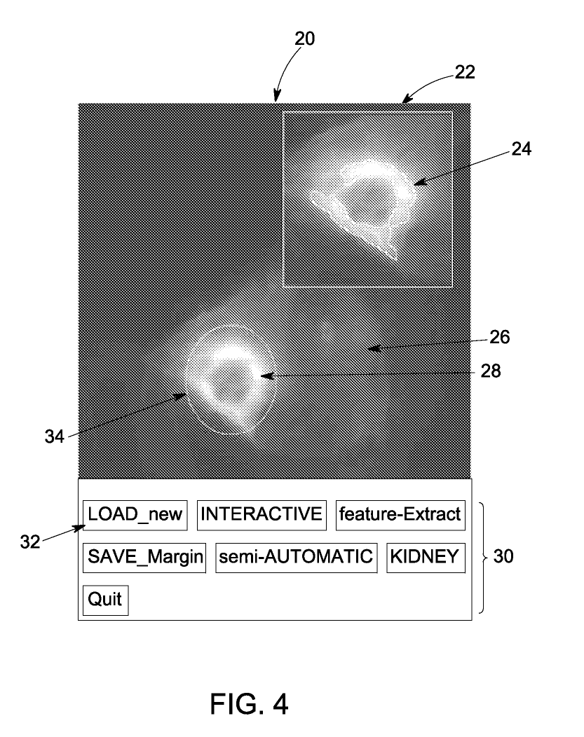

In FIG. 4, a screenshot 22 displaying an infrared image 20 is depicted. In this example, infrared image 20 depicts a tumor 28 within an organ 26, such as the skin, kidney, spleen, liver, prostate, and so forth. If the image 20 is deemed to be unsuitable, such as due to insufficient staining of the tumor 28, an operator loads a new image, such as using the “LOAD NEW” button 32 of the user input interface 30. If, however, the image 20 is deemed suitable, the operator selects the tumor 28 from the image 20, such as using a mouse, touchscreen, or other point-and-select device to select the center of the perceived tumor 28. The tumor selection process is facilitated by the display of a circle 34 or other selection area that can be centered around a point selected by the operator or which can be moved by the operator to encompass the area deemed to show the tumor 28. An automatic or semi-automatic processes can be employed, in lieu of operator input, to select the tumor 28 within the image 20. The image 20 can be enhanced, such as by implementation of anisotropic smoothing and/or other pre-processing filters.

Once the tumor 28 is selected a computer-executed algorithm automatically identifies the tumor margin 24. In one embodiment, the tumor margin 24 is identified utilizing an intensity threshold. Pixels having intensity greater than a set or threshold value can be determined to correspond to tumor tissue. In turn, those pixels determined to correspond to tumor tissue that have intensity values greater than a neighboring pixel in at least one direction can be determined to correspond to the margin 24 of the tumor 28. That is, those pixels which are stained (e.g., fluorescing) but which are adjacent to at least one other pixel that is not stained (e.g., non-fluorescing) above a certain threshold is identified as corresponding to the margin 24 of the tumor 28. Upon determination of the tumor margin 24, the circle 38 used to highlight the region having the tumor 28 is warped to highlight the identified tumor margin 24, as depicted in the inset to FIG. 4.

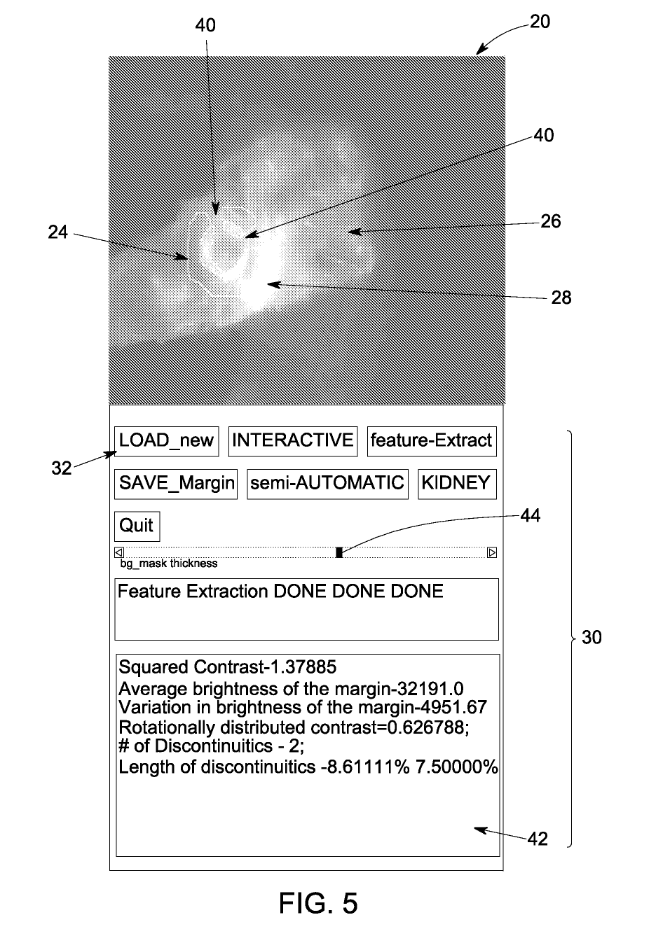

The screenshot depicted in FIG. 5, once the tumor margin 24 is identified, a computer-executed algorithm is employed to quantify one or more aspects of the tumor margin 24, such as by generating one or more margin characteristics 42, such as quantitative descriptors, of the tumor margin 24. An operator may review the margin characteristics, such as to assess the performance of the fluorescent dye used in generating the specific image 20 under review, and/or the boundary characteristics may be stored for subsequent review or comparison.

In one embodiment, the algorithm employed generates a quantitative boundary characteristics 36 i.e. squared ratio of intensities in margin to surrounding skin or squared margin to surrounding skin ratio of the tumor margin 24.

The squared average contrast is described by equation (1) as follows:

$$C = {( \frac{{I}_{margin}}{{I}_{background}} )}^{2} \qquad ( 1 )$$

where C refers to the squared average contrast, I_{margin} refers to the average pixel intensity in the tumor margin 24, and I_{background} refers to the average pixel intensity in the background region surrounding the tumor margin 24. In the depicted embodiment, the thickness of the background region used in quantifying and generating characteristics 42 such as the squared average contrast may be adjusted by the operator, such as via slider 44 of the user interface screen. Adjusting the amount or thickness of the region designated as background may vary the sensitivity and/or accuracy of the generated quantitative margin characteristics 42. In one embodiment, the background region thickness is set to a default of forty-one pixels.

The margin characteristics 42 can then be ranked, either automatically or by a reviewer, by one or more of the characteristics, allowing a reviewer to select which dyes performed best in different medical contexts, such as in different animal models, on different tumor types, based on clearance rate, and so forth.

From FIG. 1 the uptake in tumor margin versus the surrounding skin is higher for a monodisperse polylysine or discrete polylysine as indicated by higher MSR̂2 values compared to a similar molecular weight of a polydisperse polylysine.

FIG. 2 shows that for a given length of polylysine, the discrete polylysine or monodisperse polylysine (of length about 100 amino acids) is found to show higher tumor-margin to surrounding skin ratios compared to a similar length (about 122 amino acids) of polydisperse polylysine. It should be noted that in general, the MSR̂2 values increase as the length of polylysine increases.

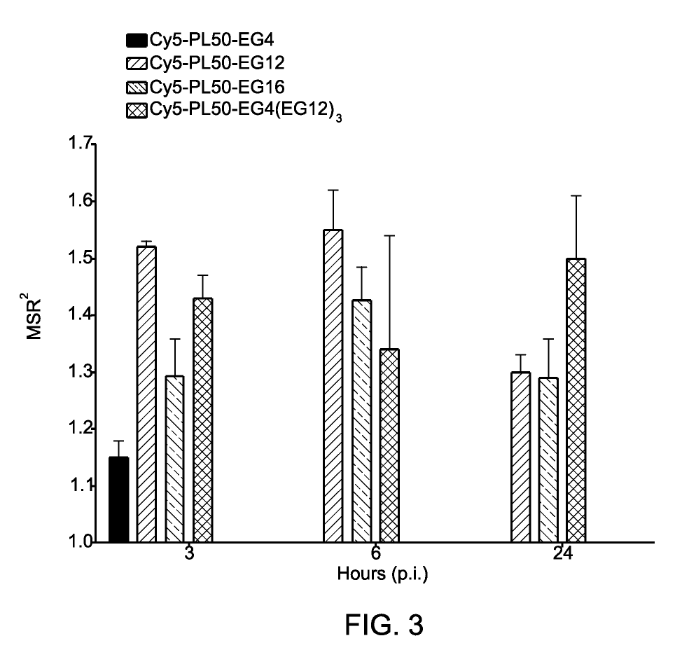

FIG. 3 shows the effect of polyethylene glycol length and structure on uptake in tumor margin and surrounding skin as well as non-specific binding at other locations. As the polyethylene glycol length increases, it takes longer for agent to accumulate in tumor margin. However, beyond certain length or if the polyethylene glycol is a branched polyethylene glycol, there is significant non-specific binding, which may be detrimental for the guided surgical procedure if the tumor margin overlaps with this non-specific region.

The foregoing examples are merely illustrative, serving to illustrate only some of the features of the invention. The appended claims are intended to claim the invention as broadly as it has been conceived and the examples herein presented are illustrative of selected embodiments from a manifold of all possible embodiments. Accordingly, it is the Applicants' intention that the appended claims are not to be limited by the choice of examples utilized to illustrate features of the present invention. As used in the claims, the word “comprises” and its grammatical variants logically also subtend and include phrases of varying and differing extent such as for example, but not limited thereto, “consisting essentially of” and “consisting of.” Where necessary, ranges have been supplied; those ranges are inclusive of all sub-ranges there between. It is to be expected that variations in these ranges will suggest themselves to a practitioner having ordinary skill in the art and where not already dedicated to the public, those variations should where possible be construed to be covered by the appended claims. It is also anticipated that advances in science and technology will make equivalents and substitutions possible that are not now contemplated by reason of the imprecision of language and these variations should also be construed where possible to be covered by the appended claims.

What is claimed is:

1. A functionalized monodisperse polylysine comprising a linear monodisperse polylysine chain comprising constituent lysine monomer residues containing appended C₄-C₂₄ polyalkylene glycol groups and at least one appended fluorescent dye moiety.

2. The functionalized monodisperse polylysine according to claim 1, wherein the polylysine molecular weight is in a range from 5000 to 250,000 Daltons.

3. The functionalized monodisperse polylysine according to claim 1, wherein the polylysine molecular weight is in a range from 10,000 to 150,000 Daltons.

4. The functionalized monodisperse polylysine according to claim 1, wherein the polylysine molecular weight is in a range from 15000 to 100,000 Daltons.

5. The functionalized monodisperse polylysine according to claim 1, wherein the constituent lysine monomer residues are greater than 90% functionalized.

6. The functionalized monodisperse polylysine according to claim 1, wherein the constituent lysine monomer residues are greater than 95% functionalized.

7. The functionalized monodisperse polylysine according to claim 1, wherein the polyalkylene glycol is a polyethylene glycol.

8. The functionalized monodisperse polylysine according to claim 1, wherein the number of fluorescent dye moieties is from about 1 to about 3 per polylysine chain.

9. The functionalized monodisperse polylysine according to claim 1, wherein the number of fluorescent dye moieties is about 1 per polylysine chain.

10. The functionalized monodisperse polylysine according to claim 1, wherein the fluorescent dye moiety is at least one selected from a phenothiazines such as methylene blue, a cyanine dye, a xanthene dye, a large Stoke shift dye, a phenoxazine dye, a benzopyrelium dye, a merocyanine dye or a styryl dye.

11. The functionalized monodisperse polylysine according to claim 1, wherein the fluorescent dye moiety is a cyanine dye.

12. The functionalized monodisperse polylysine according to claim 1, prepared by a method comprising chemical ligation.

13. A method to image a tumour margin comprising using a tumor margin imaging agent comprising the functionalized monodisperse polylysine according to claim 1.

14. A functionalized monodisperse polylysine comprising a linear monodisperse polylysine chain having a molecular weight in range from about 15,000 to 100,000 Daltons said monodisperse polylysine chain comprising constituent lysine monomer residues containing appended C₄-C₂₄ polyethylene glycol groups and at least one appended fluorescent dye moiety.

15. The functionalized monodisperse polylysine according to claim 14, wherein the number of appended polyethylene glycol groups is in a range from about 90 to about 99 per polylysine chain.

16. The functionalized monodisperse polylysine according to claim 14, wherein the number of appended polyethylene glycol groups is in a range from about 95 to about 99 per polylysine chain.

17. The functionalized monodisperse polylysine according to claim 14, wherein the number of fluorescent dye moieties is from about 1 to about 3 per polylysine chain.

18. The functionalized monodisperse polylysine according to claim 14, wherein the number of fluorescent dye moieties is about 3 per polylysine chain.

19. The functionalized monodisperse polylysine according to claim 14, wherein the fluorescent dye moiety is selected from the group consisting of a phenothiazines such as methylene blue, a phenoxazine dye, a benzopyrelium dye, a cyanine dye, a merocyanine dye or a styryl dye.

20. The functionalized monodisperse polylysine according to claim 14, wherein the fluorescent dye moiety is a cyanine dye.

21. A functionalized monodisperse polylysine comprising a linear monodisperse polylysine chain having a molecular weight in range from about 15,000 to 100,000 Daltons said monodisperse polylysine chain comprising constituent lysine monomer residues containing appended C₁₂ polyethylene glycol groups and at least one appended cyanine dye moiety.
